(19) United States
(12) Patent Application Publication
(10) Pub. No.: US 2017/0028108 A1
(43) Pub. Date: Feb. 2, 2017

(54) COATED ORTHODONTIC CRIMPABLE AUXILIARY AND METHOD FOR MAKING THE SAME

(71) Applicants: Lucas B. Karabin, Southington, CT (US); Richard W. Selander, Terryville, CT (US); Diane M. Hunter, Southington, CT (US)

(72) Inventors: Lucas B. Karabin, Southington, CT (US); Richard W. Selander, Terryville, CT (US); Diane M. Hunter, Southington, CT (US)

(21) Appl. No.: 15/219,331

(22) Filed: Jul. 26, 2016

Related U.S. Application Data
(60) Provisional application No. 62/197,223, filed on Jul. 27, 2015.
(60) Provisional application No. 62/271,769, filed on Dec. 28, 2015.

(51) Int. Cl. A61L 31/14; A61L 31/08; A61L 31/12; A61C 7/28

(52) U.S. Cl. CPC A61L 31/14; A61C 7/282; A61C 7/28; A61L 31/084; A61L 31/124

(57) ABSTRACT

An orthodontic crimpable auxiliary, having all or portions of a surface of the crimpable auxiliary configured to contact an archwire after crimping coated with diamond particles in a metal matrix. The metal matrix can be nickel.

### CROSS-REFERENCE TO RELATED APPLICATIONS

This application is a nonprovisional application claiming benefit of the filing date of provisional application 62/197,223, filed Jul. 27, 2015, and provisional application 62/271,769, filed Dec. 28, 2015, the contents of which are incorporated herein in their entirety.

### BACKGROUND OF THE INVENTION

The objective of orthodontic treatment is to reconfigure teeth in a patient's mouth to achieve a comfortable, functional bite and a more attractive smile. Orthodontists use various hardware configurations commonly known as braces to achieve this. Braces primarily consist of brackets, archwires, and auxiliaries, e.g., elastics, springs, stops and tiebacks. Together, brackets, archwires, and auxiliaries enable the orthodontist to tailor movement and repositioning of individual or segments of teeth by transferring forces to the dental facial structures.

Orthodontists have used stops on archwires since the beginning of orthodontics as a specialty in the early twentieth century. A traditional use of a stop is conventionally to prevent the archwire from sliding through a bracket beyond the desired position. This situation would cause discomfort to the patient because the end of the wire would poke out beyond the bracket into the cheek. Stops had the appearance of a V-Shaped dimple and were bent in the archwire by the orthodontist. The placement of two such stops on either side of an orthodontic bracket would prevent the archwire from sliding through the brackets and thus prevent irritation of the oral soft tissues. Two stops could also be placed on the distal side of the terminal bracket on each side of a segment of teeth to prevent space from opening in that segment, for example distal to the right and left maxillary lateral incisor brackets.

Tie backs were also bent in the archwire and were a modification of a stop. A conventional tie back loop is shown in FIG. 1. A conventional tie back loop 1 bent in archwire 2 has, as shown in FIG. 1, the appearance of a tear drop and is usually placed mesial to the first molar brackets. The tie back loop 1 can be tied to the bracket (to a hook of the bracket) with a ligature for the purpose of preventing space from opening throughout the dental arch. Tie back loops could also be placed several millimeters mesial to the first molar brackets in an archwire which also had closing loops bent in it. The tie backs could then be tied to the first molar bracket with a ligature and thereby activate the closing loops.

It is also possible to provide a tie back hook on an archwire, e.g., by soldering. A conventional tie back hook can be tied to the bracket (to a hook of the bracket) with a ligature for the purpose of preventing space from opening throughout the dental arch. The bending of stops and tie backs in an archwire is a time consuming process. The development of crimpable stops facilitated the placement of stops. An archwire could be marked with a wax marker and a small stainless steel tube, e.g., tube 3 as shown in FIG. 2, could be slid on the wire to the mark and simply crimped with an orthodontic plier to secure it in place. The placement of crimpable stops serves the same functions of preventing archwires from sliding through the brackets and preventing space from opening in a segment as the stops bent in the archwire did.

A crimpable stop having a hook is also known. See U.S. Pat. No. 5,306,142 to Richards, the contents of which are incorporated herein by reference. The Richards patent discloses an orthodontic device defining an anchor for an elastic, ligature or spring. It combines a ball hook attached to a crimpable stop, the stop having a tubular body with inner wire-engaging surfaces coated with an abrasive material to produce an anti-sliding and anti-rotating engagement with the wire when crimped down upon the wire. This patent discloses that the crimpable ball hooks can be mounted on archwires and when used with intermaxillary elastics can provide intermaxillary fixation, can be mounted on a lip bumper received in buccal tubes or can be mounted on the inner bow of a face bow received in buccal tubes.

A schematic view of a crimpable hook having a hook 4 connected to a crimpable tube 5 having a split body is shown in FIG. 3.

U.S. Pat. No. 5,306,142 to Richards also discloses that an abrasive coating may be applied to all or part of the inner surface of the crimpable stops to engage the archwire when the hook or stop is crimped, thereby increasing the friction between the hook or stop and the wire, after crimping. The abrasive coating which is applied to all or selected portions of the inner surface of the crimpable body may be a layer of suitable carborundum and may be applied by a suitable coating device.

### SUMMARY OF THE INVENTION

The present invention relates to an orthodontic crimpable auxiliary, having all or portions of a surface of the crimpable auxiliary configured to contact an archwire after crimping coated with diamond particles in a metal matrix. The metal matrix can be nickel.

### BRIEF DESCRIPTION OF THE DRAWINGS

FIG. 1 is a schematic view of a conventional tie back loop.

FIG. 2 is a schematic view of a crimpable tube.

FIG. 3 is a schematic view of a crimpable hook.

FIG. 4 is a schematic perspective view of a crimpable auxiliary applicant has developed as set forth in U.S. provisional patent application No. 62/197,223, filed Jul. 27, 2015 and the nonprovisional claiming benefit thereof filed on Jul. 26, 2016 (Attorney Docket No. 20394-139303-US).

FIG. 5A is a graph showing pull tests for uncoated crimp stops on nickel titanium (NiTi) archwires.

FIG. 5B is a graph showing pull tests for crimp stops coated with diamond particles contained in a nickel matrix on nickel titanium (NiTi) archwires.

FIG. 6A is a graph showing pull tests for uncoated crimp stops on beta titanium archwires.

FIG. 6B is a graph showing pull tests for crimp stops coated with diamond particles contained in a nickel matrix on beta titanium archwires.

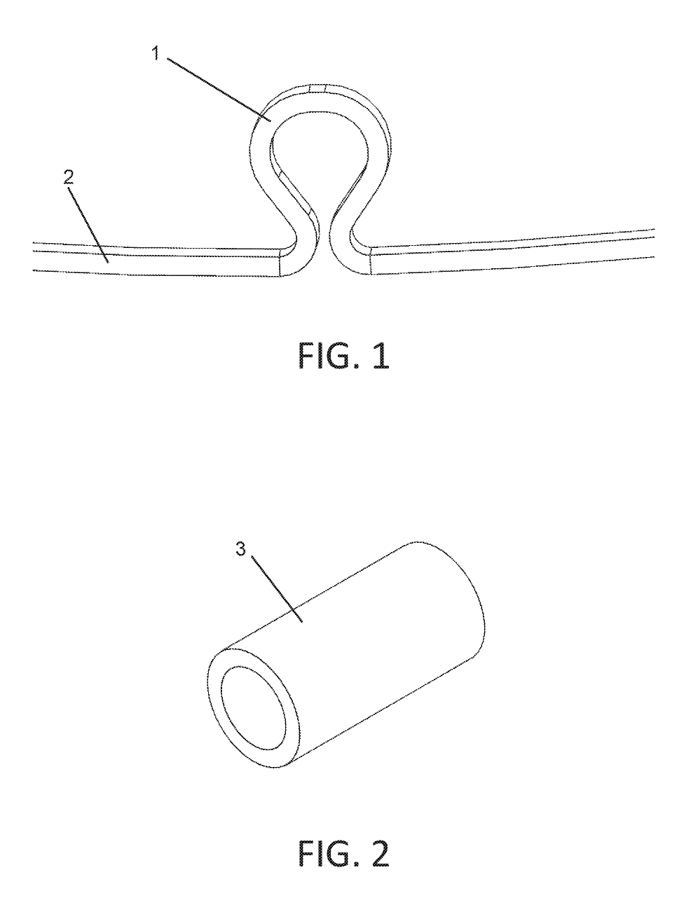

### DETAILED DESCRIPTION OF THE PREFERRED EMBODIMENTS

Applicant has found that it would be advantageous to provide a more efficient friction increasing and biocompatible coating on the inner walls of crimpable stops to provide an increased anti-sliding effect.

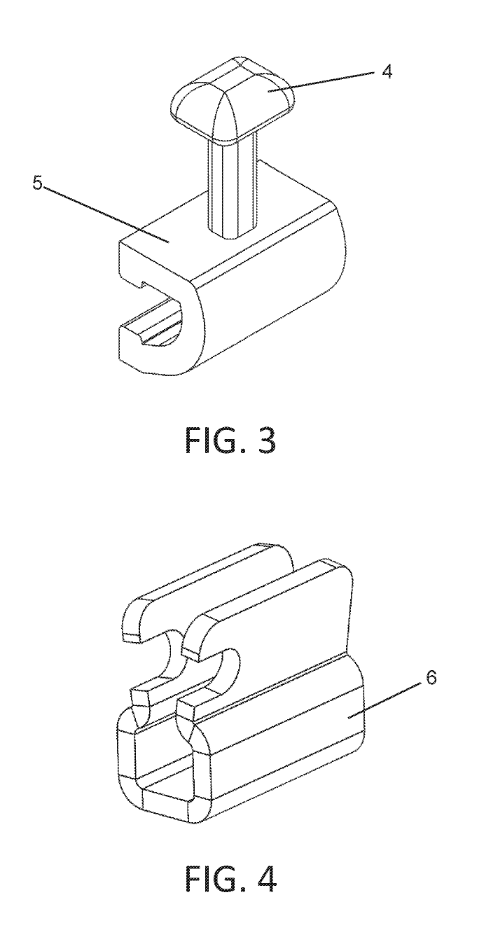

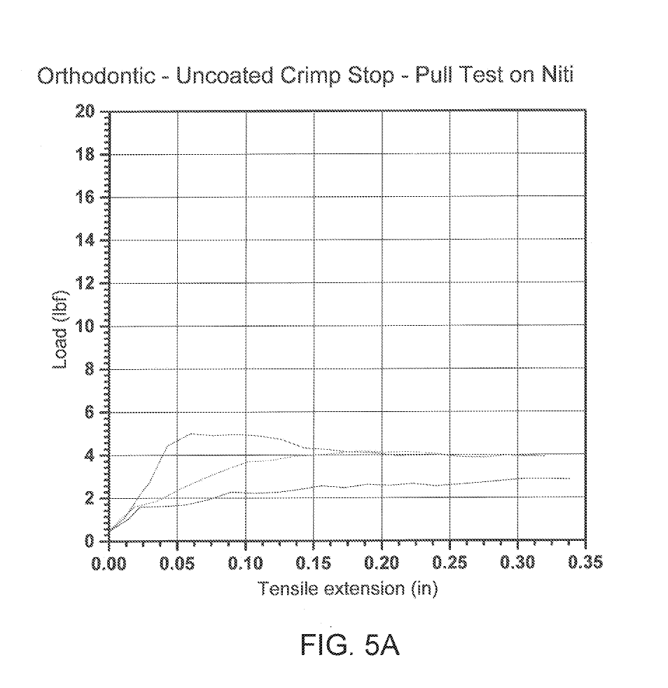

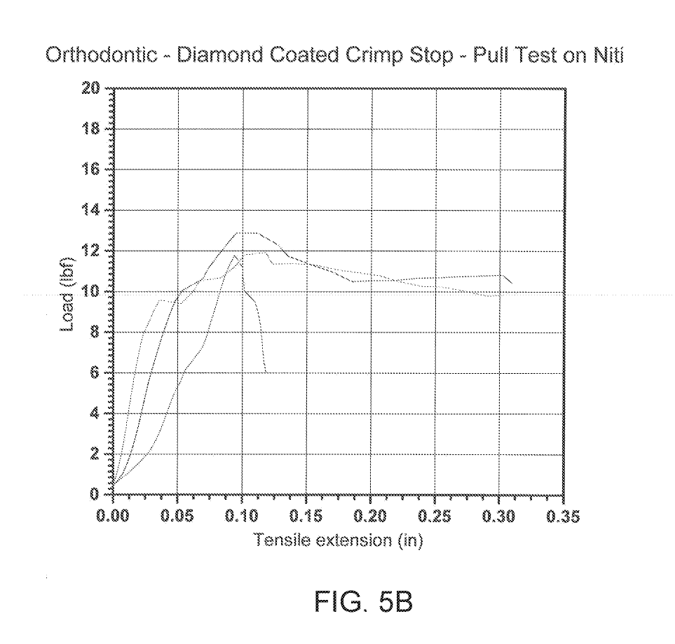

The present invention is useful for any type of crimpable auxiliary, including crimpable stops or crimpable hooks, etc., including but not limited to completely closed tubes (see, e.g., FIG. 2), crimpable auxiliaries having a split body (see, e.g., FIG. 3), crimpable auxiliaries without and with hooks or ball hooks (see, e.g., FIG. 3), and crimpable auxiliary applicant has developed with a wing joined to the tubular shaped portion of the stop and having a slot provided in the wing open to an end of the wing in the mesial or distal direction and having a depth in the mesial distal direction and a height in a gingival-occlusal direction configured to receive a ligature wire or elastic, as set forth in U.S. provisional patent application No. 62/197,223, filed Jul. 27, 2015 and the nonprovisional claiming benefit thereof filed on Jul. 26, 2016 (Attorney Docket No. 20394-139303-US), the contents of each of which are incorporated herein by reference; such a crimpable auxiliary 6 is shown in FIG. 4.

According to the present invention, the friction increasing coating is applied to all or portions of the inner surface of the crimpable stop that contact the archwire after crimping.

According to the present invention, the coating is one having diamond particles contained in a biocompatible metal layer matrix. One example of such a diamond coating is the coating sold by Surface Technology, Inc. of Robbinsville, N.J. under the trademark Composite Diamond Coating®. That diamond coating has ultra-fine diamond particles contained within hard electroless nickel metal. Such a coating and methods for making and using such a coating are described in U.S. Pat. Nos. 4,997,686, 5,145,517, 5,300,330, 5,863,616, 6,306,466, 7,744,685 and 8,147,601, the contents of each of which are incorporated herein by reference.

The coating of the present invention preferably has an electroless nickel matrix and diamond particles in the matrix. Such a coating provides benefits including high wear resistance, hardness and corrosion resistance. It conforms to complex geometries so that it can be applied to all or portions of the inner surface of the crimpable stop that contact the archwire after crimping. It is applicable to all common metals and alloys and therefore can be applied to alloys commonly used for crimpable stops.

The coating of the present invention, i.e., a coating having diamond particles contained in a biocompatible metal layer matrix, is biocompatible and therefore can be used on orthodontic appliances. Because of the chemical inertness and hardness of industrial diamond particles and that the coating is highly resistant to corrosion and abrasion, there is little or no likelihood that components of this material would separate from the auxiliary and be swallowed by the patient.

Another advantage of the coating used in the present invention is its high wear resistance as compared to coatings of many other materials.

In the coating of the present invention, the density of diamond particles within the metal matrix is typically 25-40% by volume. Higher and lower densities can be used but too little diamond in the metal matrix will not sufficiently provide the properties of diamond desired in the coating and too much diamond in the metal matrix would reduce the amount of the metal matrix required for adhesion to the inside surface of the crimpable stop and reduce structural integrity of the coating.

The diamond particles have a mean particle size of 0.1-50 microns, preferably 2-10 microns, most preferably 8 microns, i.e., CDC-8.

The coating can be applied to all or portions of the inner surface of the crimpable auxiliary that contact the archwire after crimping by the process described in U.S. Pat. Nos. 4,997,686, 5,145,517, 5,300,330, 5,863,616, 6,306,466, 7,744,685 and 8,147,601, the contents of each of which are incorporated herein by reference. That is, the inner surface of the crimpable stop can be electrolessly metallized with a metal coating incorporating diamond particulate matter therein. The process comprises contacting the inner surface of the crimpable stop with a stable electroless metallizing bath comprising, inter alia, a metal salt, an electroless reducing agent, and a quantity of diamond particulate matter which is essentially insoluble or sparingly soluble in the metallizing bath, and maintaining the diamond particulate matter in suspension in the metallizing bath during the metallizing of the inner surface of the crimpable stop for a time sufficient to produce a metallic coating with the diamond particulate matter dispersed therein.

Areas of the crimpable auxiliary which are desired to not be coated with the coating can be masked prior to the electroless plating. However, in view of the small size of the crimpable auxiliaries and the fact the coating does not irritate any surfaces of the oral cavity in which it might come in contact, it is more simple and cost-efficient to coat all exposed surfaces.

The coating is applicable to all common metals and alloys and therefore can be applied to alloys commonly used for crimpable auxiliaries with a suitable cleaning, activation and pretreatment process for the metal or alloy used as the substrate, as would be understood by those skilled in the art based.

The crimpable auxiliaries can be used for all known archwire materials, e.g., stainless steel, NiTi alloys, Cu—NiTi alloys, cobalt-chromium alloys, beta-titanium, etc.

### Examples

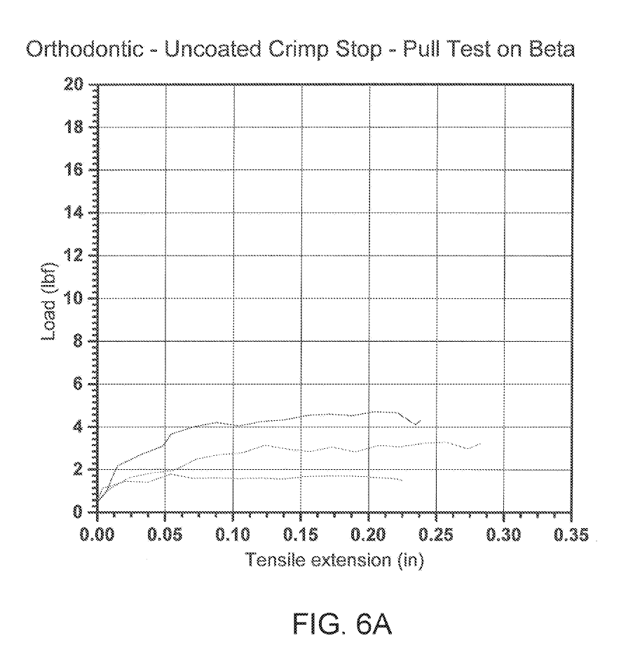

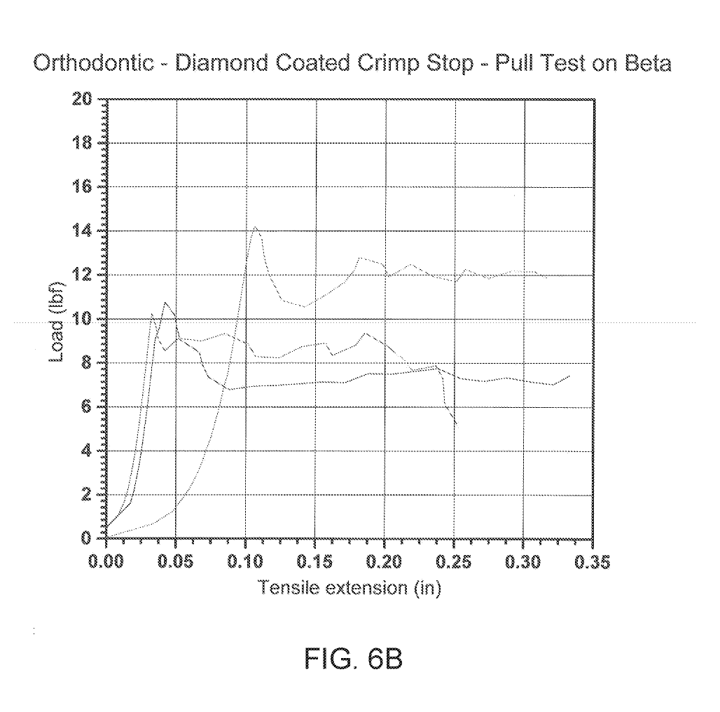

FIG. 5A is a graph showing pull tests for three uncoated crimp stops crimped on three nickel titanium (NiTi) archwires. FIG. 5B is a graph showing pull tests for three crimp stops coated with diamond particles contained in a nickel matrix crimped on three nickel titanium (NiTi) archwires. FIG. 6A is a graph showing pull tests for three uncoated crimp stops crimped on three beta titanium archwires. FIG. 6B is a graph showing pull tests for three crimp stops coated with diamond particles contained in a nickel matrix crimped on three beta titanium archwires.

For these tests, substantially identical crimpable stops were provided. One set of the crimpable stops were left uncoated, i.e., the inner surfaces of the “uncoated crimp stops” were not coated with a friction increasing coating. Another set of the crimpable stops were fully coated with a coating having diamond particles contained in a biocompatible metal layer matrix. More particularly, the “diamond coated crimp stops” were coated with a coating having diamond particles of size CDC-8 (mean particle size of 8 microns) in a nickel matrix. The coating is sold under the trademark Composite Diamond Coating® by Surface Technology, Inc. of Robbinsville, N.J.

The crimp stops were crimped onto the archwires and a pull test conducted on each of the crimp stop/archwire combination on an Instron® load tester. The archwire was put through a hole large enough for the archwire to pass through but too small for the crimp stop to pass. The archwire was held at the opposite end in a jaw similar to a vice grip. The test specimen was then pulled and the pull force, load in lbf., was measured at various extension increments. When the curve peaked, it is where the crimp stop lets loose and starts to slip along the wire. The results are shown in FIGS. 5A, 5B, 6A and 6B.

As can be seen from FIGS. 5A, 5B, 6A and 6B, the load required before the archwire slipped through the crimp stop was much higher, i.e., over 9 to approximately 13 lbf for nickel titanium (NiTi) archwires (FIG. 5B) and approximately 10-14 lbf for beta titanium archwires (FIG. 6B) for crimp stops coated with diamond particles contained in a nickel matrix than for uncoated crimp stops, i.e., less than 2 to approximately 5 lbf for nickel titanium (NiTi) archwires (FIG. 5A) and less than 2 to approximately 4 lbf for beta titanium archwires (FIG. 6A). This demonstrates a large increase in the friction providing function for the crimp stops coated with diamond particles contained in a metal matrix.

Without being held to any particular theory, the inventors theorize that the diamond particles may “bite” into the archwire when the crimpable auxiliary is crimped on the archwire, leading to the large increase in the load required before the archwire slipped through the crimpable auxiliary.

What is claimed is:

1. An orthodontic crimpable auxiliary, having all or portions of a surface of the crimpable auxiliary configured to contact an archwire after crimping coated with diamond particles in a metal matrix.

2. The orthodontic crimpable auxiliary according to claim 1, wherein the metal matrix is nickel.

3. The orthodontic crimpable auxiliary according to claim 1, wherein the auxiliary is a crimpable stop.

4. The orthodontic crimpable auxiliary according to claim 1, wherein the auxiliary is a crimpable hook.
